(19) United States
(12) Patent Application Publication
(10) Pub. No.: US 2026/0130697 A1
(43) Pub. Date: May 14, 2026

(54) INTRAMEDULLARY NAIL

(71) Applicant: CITIEFFE S.R.L., Calderara di Reno (IT)

(72) Inventors: Alan DOVESI, Calderara di Reno (IT); Massimiliano MORETTI, Calderara di Reno (IT)

(21) Appl. No.: 19/385,611

(22) Filed: Nov. 11, 2025

(30) Foreign Application Priority Data
Nov. 12, 2024 (IT) 102024000025521

(51) Int. Cl. A61B 17/72

(52) U.S. Cl. CPC A61B 17/7241; A61B 17/7266

(57) ABSTRACT

Intramedullary nail for insertion into the medullary canal of long bones, comprising a first anchoring portion at its distal end, having at least one transverse through hole for allowing the passage of a first locking screw, a shank extending longitudinally from said first anchoring portion, a second anchoring portion at its proximal end longitudinally opposite to the first anchoring portion.

This application claims priority to Italian Patent Application 102024000025521 filed Nov. 12, 2024, the entirety of which is incorporated by reference herein.

The present invention relates to an intramedullary nail.

In particular, the present invention relates to an intramedullary nail designed for the treatment of long bone fractures.

By way of example and without limitation, the following discussion will refer to fractures of the fibula.

Such fractures, which are common in traumatology, may require surgery to stabilize the bone and allow it to heal properly.

Intramedullary nails are widely used to ensure stable and minimally invasive internal fixation. However, one of the main problems encountered in the current technique concerns the fixation of the proximal end of the nail to the bone.

Known types of intramedullary nails for the fibula involve proximal locking systems using transverse screws, radial expansions, or wedge mechanisms.

Although these solutions represent an advance in orthopedic surgery, they have significant limitations, especially when applied to the fibula.

In particular, transverse screws are commonly used to ensure the stability of the proximal end of the nail, but the irregular shape of the fibula, combined with its small size in the proximal area, makes it difficult to achieve adequate and secure locking.

Inserting transverse screws into the fibula can be technically complex, resulting in suboptimal fixation that leads to micro-movements and instability over time. These movements can compromise bone healing, delay recovery, and, in some cases, require corrective surgery.

Other known approaches, such as radial expansion systems or wedge mechanisms, while improving load distribution, can be difficult to insert and do not always guarantee secure fixation in small bones such as the proximal fibula.

Furthermore, overly aggressive expansion mechanisms can cause damage to bone tissue or create unwanted stress in the area.

The key issue concerns the ability to achieve stable and durable fixation of the proximal end of the nail, especially in a relatively small and delicate bone such as the fibula, where the space available for screws or other locking systems is limited. The small size of the proximal portion of the fibula increases the risk of fixation failure, with possible consequences such as loosening of the nail, bone misalignment, and slowing of the healing process.

The present invention aims to solve the problems associated with fixing the proximal end of the nail for the treatment of fibula fractures, overcoming the limitations of existing technologies.

In particular, the object of the present invention is to provide an intramedullary nail for the fibula provided with a locking system that ensures greater stability and resistance than currently available solutions, optimized for the anatomical conformation of the proximal fibula and its small size.

A further object of the present invention is to provide an intramedullary nail that simplifies the surgical procedure and promotes faster and safer healing.

The technical characteristics of the invention, according to the above objects, are clearly described in the attached claims, and its advantages are evident from the detailed description that follows, with reference to the attached drawings that illustrate an exemplary and non-limiting embodiment, wherein:

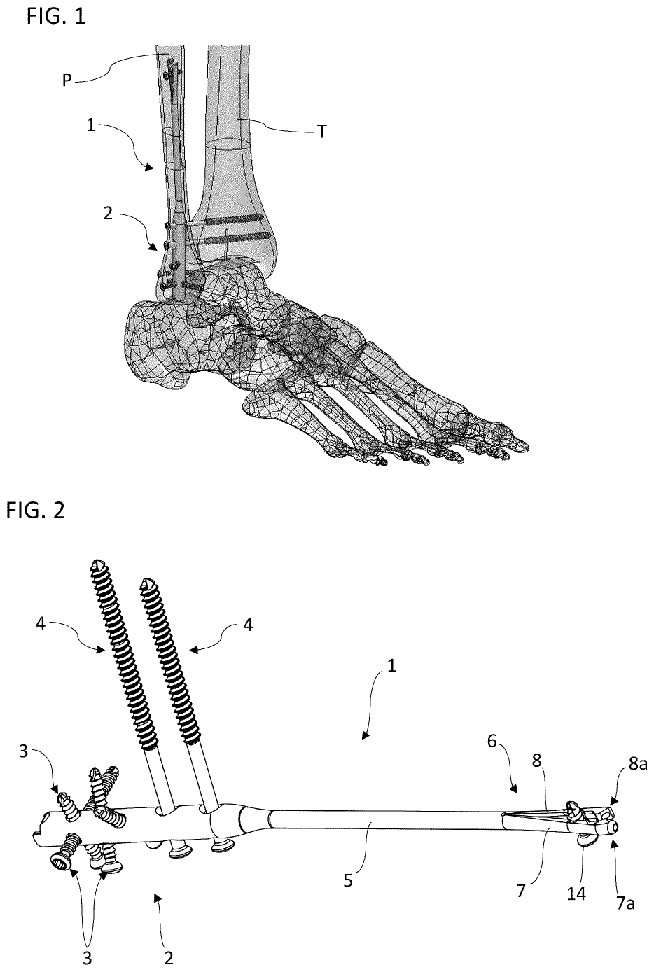

FIG. 1 illustrates, in a schematic perspective view, with transparent parts resembling an X-ray, an intramedullary nail according to the present invention applied to a patient;

FIG. 2 illustrates, in a schematic perspective view, the intramedullary nail according to the present invention with some screws suitable for its implantation;

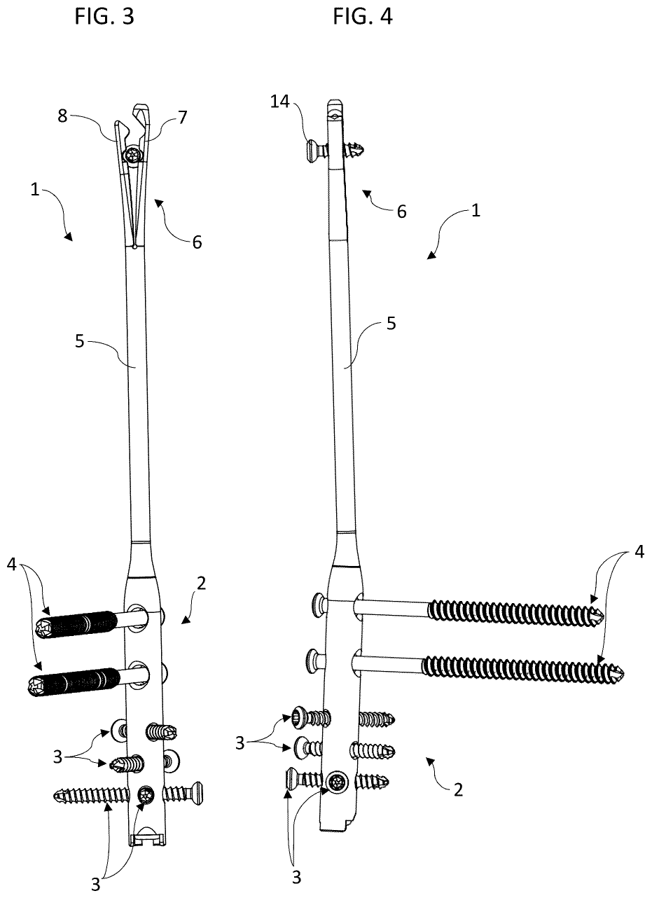

FIGS. 3 and 4 illustrate, in respective schematic perspective views from different angles, the intramedullary nail referred to in the previous figures with some screws suitable for its implantation;

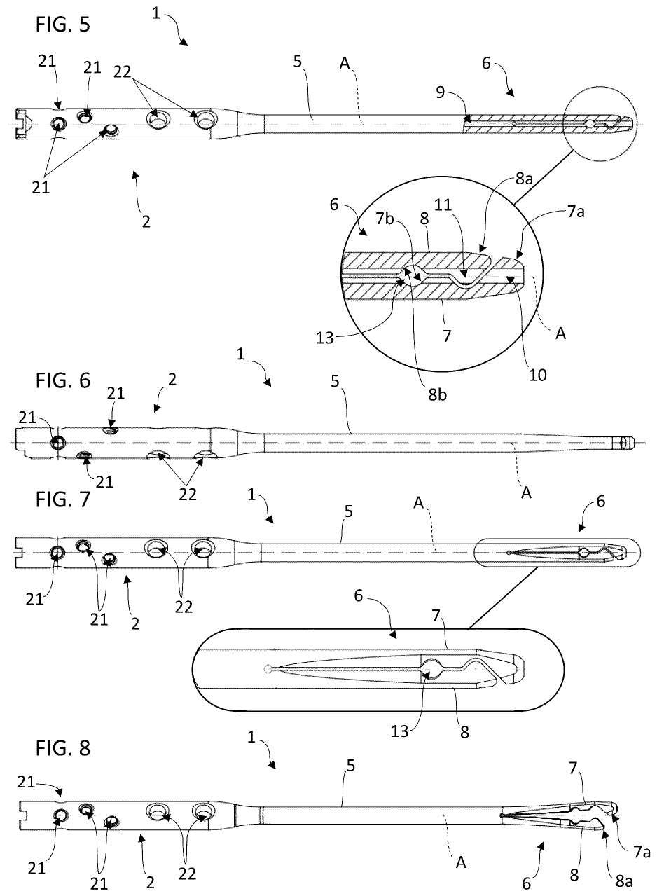

FIG. 5 illustrates, in a schematic side elevation view with some parts in section, the intramedullary nail according to the present invention, in a first phase of its implementation;

FIGS. 6 and 7 illustrate, in respective schematic views in different elevations, the intramedullary nail referred to in FIG. 5;

FIG. 8 illustrates, in a schematic side elevation view, the intramedullary nail according to the present invention in a second phase of its implementation;

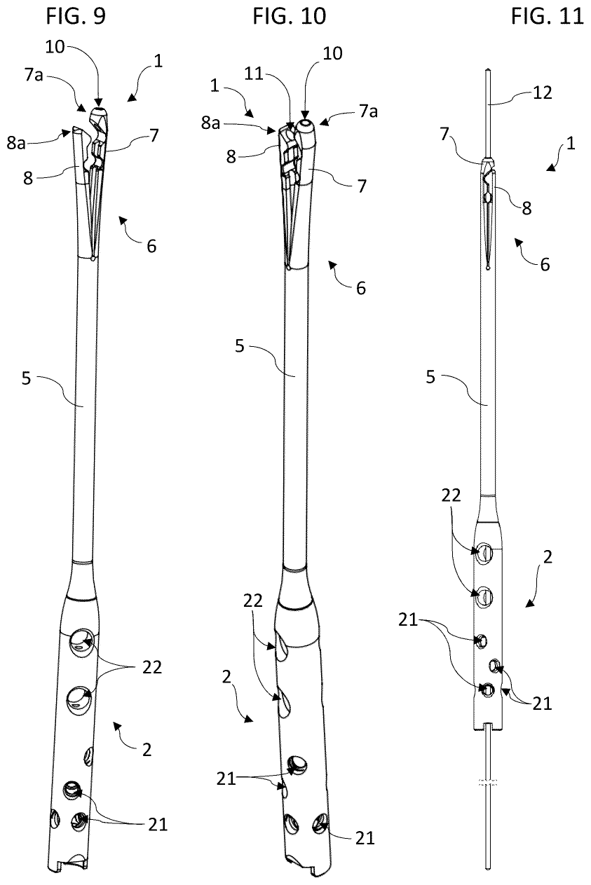

FIGS. 9 and 10 illustrate, in respective schematic perspective views from different angles, the intramedullary nail referred to in FIG. 8;

FIG. 11 shows, in a schematic elevation view, the intramedullary nail according to the present invention in a configuration of use adopted during its implantation in the fibula of a patient.

As illustrated in FIG. 1, reference number 1 illustrates an intramedullary nail for insertion into the medullary canal of long bones made in accordance with the present invention, said nail 1 being exemplarily illustrated implanted in the fibula of a patient.

As shown in FIG. 1, the intramedullary nail 1 according to the invention is designed to be inserted into the medullary canal of the fibula P and comprises a first anchoring portion 2 located at its distal end.

For the sake of brevity, in the remainder of this discussion, the intramedullary nail 1 for insertion into the medullary canal of the fibula will also be referred to simply as nail 1 or intramedullary nail 1.

As better illustrated in FIGS. 5 to 10, in the aforementioned first anchoring portion 2, the nail 1 has a plurality of first transverse through holes 21 designed to allow the passage of respective first locking screws 3 visible in FIG. 2.

The first locking screws 3 are designed to engage with the patient's fibula P.

Again with reference to FIGS. 5 to 10, in the aforementioned first anchoring portion 2, the nail 1 has two second transverse through holes 22 designed to allow the passage of respective second locking screws 4 visible in FIG. 1.

The second holes 22 are arranged in a more central position along the longitudinal length of the nail 1 relative to the first holes 21.

The second locking screws 4 are designed to screw into the patient's tibia T.

The nail 1 comprises a shank 5 extending longitudinally from the aforementioned first anchoring portion 2 and developing proximally with respect to it.

As illustrated in FIGS. 1 and 2, the nail 1 comprises a second anchoring portion 6 at its proximal end longitudinally opposite the aforementioned first anchoring portion 2.

As is standard practice in the medical field, the distal and proximal indications referring to parts of the nail 1 are to be understood with respect to the center of the body of a patient in whom the nail 1 itself is implanted.

As illustrated in particular in FIGS. 8 to 10, the nail 1 has, at its second anchoring portion 6 located in the proximal end area, two prongs 7 and 8, respectively a first and a second prong.

As clearly illustrated in FIGS. 8 to 10, the two prongs 7 and 8 are normally spread apart so that they expand elastically away from each other when brought together.

In other words, as will be described in more detail below, starting from a close configuration, which has been forcibly imposed on it (such as that illustrated in FIG. 11, for example), the two prongs 7, 8 tend to naturally assume the spread configuration illustrated in FIGS. 8 to 10.

Operationally, starting from a semi-finished product (not illustrated) of nail 1, the two prongs 7, 8 are obtained, at a proximal portion of nail 1 that is still substantially cylindrical, advantageously by means of wire electrical discharge machining (wire EDM).

In other words, during the early stages of processing, and before the two prongs 7, 8 are made, the nail 1 is basically axially symmetrical, although with sections of varying diameter along its length.

The nail 1 has, inside it, a first cylindrical cavity 9, partially visible in FIG. 5, suitable for the passage of a metal wire.

The aforementioned first cylindrical cavity 9 extends longitudinally both along the first anchoring portion 2 and along the shank 5, ending up facing the aforementioned second proximal anchoring portion 6.

The first cylindrical cavity 9 has a central axis of development A.

As shown in FIGS. 5, 9, and 10, at their proximal ends, the two prongs 7, 8 have respective second 10 and third 11 cylindrical cavities.

Both the second and third cylindrical cavities 10, 11 are through cavities.

To obtain the aforementioned second 10 and third 11 cylindrical cavities, the prongs 7 and 8 have respective proximal end portions 7a and 8a that are counter-shaped to interlock with respect to the aforementioned central axis A.

In other words, in the configuration of nail 1 with prongs 7 and 8 brought closer together, as illustrated in FIGS. 5 to 7, the two respective proximal end portions 7a, 8a are designed to position themselves in such a way as to align the respective central axes of their second and third cylindrical cavities 10, 11 with each other and aligned with the central axis A of the first cylindrical cavity 9.

The alignment of the three cylindrical cavities 9, 10, and 11 with respect to axis A, visible in the enlarged circled image in FIG. 5, therefore defines a continuity between the cavities themselves and allows a metal wire 12 to pass through them, as illustrated in FIG. 11.

As shown in detail in FIGS. 5 and 7, a gap 13 in the material is created between the two prongs 7, 8.

The gap 13 is defined by two curved walls 7b, 8b, one on each of the prongs 7, 8, facing each other.

The gap 13 in the material created between the prongs 7, 8 defines a housing designed to engage a third screw 14 for locking the nail 1.

The third locking screw 14 is therefore screwed into the gap 13 and increases and maintains the spacing between the prongs 7, 8.

As already mentioned above, the two prongs 7, 8 are advantageously obtained by wire EDM.

The result of this electroerosion phase is illustrated in FIGS. 5 to 7, in which the two prongs 7, 8 are defined by the formation, by electroerosion, of a gap between them.

Similarly, the gap 13 in the material is advantageously obtained by wire electroerosion.

Essentially, in a manner not illustrated, once the two prongs 7, 8 have been obtained by electroerosion, they are spread apart by plastic deformation, so that this spread configuration, as illustrated in FIGS. 9 and 10, is permanent.

When, in practical use, the two prongs 7, 8 are forced to assume the approximate configuration shown in FIG. 11, they maintain it only by virtue of a physical impediment such as that represented by the metal wire 12 inserted inside the nail 1 in its first cylindrical cavity 9 and passing through the additional cylindrical cavities 10, 11 of both prongs 7, 8.

As illustrated in FIGS. 9 and 10, the respective inner faces of the prongs 7, 8 facing each other have respective channels that act as guides for the metal wire 12 to slide along the section of nail 1 defined by the prongs 7, 8.

These channels derive from the division into two halves, following the electroerosion phase, of the portion of the first cylindrical cavity 9 that extended at the second anchoring portion 6, precisely before the electroerosion phase.

The metal wire 12, together with the second and third cylindrical cavities 10, 11, defines removable means for fixing the prongs 7, 8 to the nail 1, which removable securing means are configured to hold the prongs 7, 8 in close proximity and, once removed, to allow their elastic expansion, meaning their spreading apart.

Starting from the configuration shown in FIG. 11, the extraction of the wire 12 from the second and third cylindrical through cavities 10, 11 therefore determines the removal of the physical impediment that holds the two prongs 7, 8 close together, with the simultaneous elastic release of the same, which tend to spread apart to assume the configuration illustrated in FIGS. 8 to 10.

This is what happens during surgery when the surgeon has positioned the nail 1 inside the medullary cavity of the fibula P and can therefore proceed to remove the metal wire 12.

When the nail 1 is inserted into the medullary cavity of the fibula P, therefore, also due to the actual dimensions of the latter, it may be advantageous not to complete the spreading, thereby causing the continuation of an elastic forcing action on the internal cortical wall of the fibula P.

This elastic action against the inner cortical wall of the fibula P already contributes to the stabilization of nail 1 with respect to the fibula P itself.

In practice, this is a sort of wedging of the intramedullary nail 1 against the inner cortical wall of the fibula P.

To improve and, in any case, ensure greater anchoring of prongs 7 and 8 against the inner cortical wall of the fibula P, the surgeon inserts the third locking screw 14 into the gap 13 between prongs 7 and 8.

The curvature of the aforementioned curved walls 7b, 8b is such as to allow the thread of the third screw 14 to be screwed in, which, during screwing, by interposing itself between the two prongs 7, 8, contributes to their separation, possibly accentuating the force exerted by the proximal portions 7a, 8a of the ends of the prongs 7, 8 on the inner cortical wall of the fibula P.

The third screw 14 also defines a physical impediment to the prongs 7, 8 abandoning their spread configuration.

The intramedullary nail 1 according to the invention achieves the set objects and offers significant advantages.

A first advantage associated with the intramedullary nail according to the invention is that its proximal anchoring portion has a very small footprint, thereby facilitating the insertion of the nail itself into the medullary cavity.

A further advantage associated with the nail according to the invention is that the prongs formed at the proximal anchoring portion already ensure increased proximal stability of the nail.

Another advantage of the nail according to the invention is the possibility of inserting a screw between the two prongs in order to adjust the pressure with which the prongs engage against the inner cortical wall of the fibula, thereby allowing optimal locking of the nail at its proximal end.

Another advantage associated with the intramedullary nail according to the invention lies in the fact that the simple removal of the screw between the two prongs facilitates the possible extraction of the nail.

1. An intramedullary nail for insertion into the medullary canal of long bones, comprising:
a first anchoring portion at a relative distal end area, comprising at least a plurality of first transversal through holes designed to allow the passage of first locking screws,
a shank extending longitudinally from said first anchoring portion,
a second anchoring portion at the relative proximal end area longitudinally opposite said first anchoring portion, the second anchoring portion having two prongs (7, 8) configured to elastically expand away from each other starting from a close configuration, said first anchoring portion and shank presenting therein a first cylindrical cavity facing said second anchoring portion, wherein it comprises removable means of fixing said prongs, configured for keeping said prongs in a close position and, once removed, allow the elastic expansion of the prongs themselves.

2. The intramedullary nail according to claim 1, wherein said removable fixing means comprise second and third cylindrical cavities respectively formed in said two prongs, said second and third cylindrical cavities being aligned, with each other and with said first cylindrical cavity, when said prongs are in a close configuration.

3. The intramedullary nail according to claim 2, wherein said removable means for fixing said prongs comprise a metal wire slidable in said first cylindrical cavity and designed to engage simultaneously inside said second and third cylindrical cavities aligned in said close configuration of said prongs.

4. The intramedullary nail according to claim 3, wherein said metal wire is slidable in said first, second and third cylindrical cavities to be extracted from said second and third cylindrical cavities and allow the elastic expansion of said prongs starting from said close configuration thereof.

5. The intramedullary nail according to claim 1, wherein it has a gap of material between said prongs when close to each other.

6. The intramedullary nail according to claim 5, wherein said gap is defined by two curved walls formed respectively on said prongs and facing each other.

7. The intramedullary nail according to claim 6, wherein said gap is configured to define a housing designed to engage a screw for locking the nail itself.
